(19) United States
(12) Patent Application Publication
(10) Pub. No.: US 2024/0115467 A1
(43) Pub. Date: Apr. 11, 2024

(54) COSMETIC PREPARATION

(71) Applicant: SHISEIDO COMPANY, LTD., Chuo-ku, Tokyo (JP)

(72) Inventors: Megumi SUGIHARA, Tokyo (JP); Ryo FURUKAWA, Tokyo (JP); Kayoko NAOI, Tokyo (JP); Chi Je PARK, Tokyo (JP)

(73) Assignee: SHISEIDO COMPANY, LTD., Chuo-ku, Tokyo (JP)

(21) Appl. No.: 18/275,563

(22) PCT Filed: Feb. 22, 2022
(86) PCT No.: PCT/JP2022/007103

§ 371 Date: Aug. 2, 2023

(30) Foreign Application Priority Data
Mar. 2, 2021 (JP) 2021-032671

(51) Int. Cl. A61K 8/27; A61K 8/06; A61Q 1/00

(52) U.S. Cl. CPC A61K 8/27; A61K 8/06; A61Q 1/00

(57) ABSTRACT

A cosmetic having an excellent effect of solidifying sebum is to be provided.

A cosmetic is used that includes low aspect ratio zinc oxide having an average aspect ratio of less than 4 and high aspect ratio zinc oxide having an average aspect ratio of 4 or more, wherein
an amount of the high aspect ratio zinc oxide blended is 1 to 80 mass % with respect to a total amount of the high aspect ratio zinc oxide and the low aspect ratio zinc oxide.

### TECHNICAL FIELD

The present invention relates to a cosmetic including zinc oxide.

### BACKGROUND ART

Sebum secreted from the skin mixes with a component of a makeup cosmetic, and causes the makeup to smear. When a large amount of sebum is secreted, the sebum comes to the surface of the face, and causes a sebum smear. As a measure against a sebum smear, it is known to blend a powder containing zinc oxide or the like that solidifies sebum and suppress a sebum smear by solidifying sebum (for example, see Patent Literature 1).

For zinc oxide to be blended in cosmetics, specific ranges of the surface treatment, the average particle size, the specific surface area, and the like have been studied. However, as far as the present inventor knows, there is no report on a combination in which zinc oxide has a specific aspect ratio.

Users have high desire for suppressing a sebum smear, and development of a cosmetic that can further suppress a sebum smear has been awaited.

### CITATION LIST

### Patent Literature

Patent Literature 1: JP 2020-33274 A

### SUMMARY OF THE INVENTION

The present inventors have surprisingly found that a cosmetic including low aspect ratio zinc oxide and high aspect ratio zinc oxide wherein the amount of the high aspect ratio zinc oxide blended is 1 to 80 mass % with respect to the total amount of the high aspect ratio zinc oxide and the low aspect ratio zinc oxide can realize a cosmetic excellent in an effect of solidifying sebum and capable of suppressing smearing of makeup. The present invention is based on these findings.

Accordingly, the present invention discloses a cosmetic including low aspect ratio zinc oxide and high aspect ratio zinc oxide.

According to the present invention, the following invention is provided.

(1) A cosmetic including:
low aspect ratio zinc oxide having an average aspect ratio of less than 4; and high aspect ratio zinc oxide having an average aspect ratio of 4 or more, wherein
an amount of the high aspect ratio zinc oxide blended is 1 to 80 mass % with respect to a total amount of the high aspect ratio zinc oxide and the low aspect ratio zinc oxide.

(2) The cosmetic according to (1), wherein the low aspect ratio zinc oxide is zinc oxide surface-treated with an alkylalkoxysilane.

(3) The cosmetic according to (1) or (2), wherein the low aspect ratio zinc oxide is zinc oxide surface-treated with octyltriethoxysilane.

(4) The cosmetic according to any one of (1) to (3), wherein the low aspect ratio zinc oxide has an average particle size of 20 to 60 nm.

(5) The cosmetic according to any one of (1) to (4), wherein the low aspect ratio zinc oxide has an average particle size of 30 to 55 nm.

(6) The cosmetic according to any one of (1) to (5), wherein the high aspect ratio zinc oxide is a plate-shaped crystal.

(7) The cosmetic according to any one of (1) to (6), further including a dispersant.

(8) The cosmetic according to any one of (1) to (7), being a water-in-oil or oil-in-water emulsion cosmetic.

The cosmetic according to the present invention has an excellent effect of solidifying sebum and can suppress smearing of makeup.

### BEST MODE FOR CARRYING OUT THE INVENTION

The present invention relates to a cosmetic including low aspect ratio zinc oxide having an average aspect ratio of less than 4 (hereinafter, also simply referred to as “low aspect ratio zinc oxide”) and high aspect ratio zinc oxide having an average aspect ratio of 4 or more (hereinafter, also simply referred to as “high aspect ratio zinc oxide). In the present cosmetic, the amount of the high aspect ratio zinc oxide blended is 1 to 80 mass %, preferably 1 to 60 mass %, more preferably 3 to 40 mass %, and still more preferably 10 to 30 mass % with respect to the total amount of the high aspect ratio zinc oxide and the low aspect ratio zinc oxide.

The effect of solidifying sebum is surprisingly improved by combination of the low aspect ratio zinc oxide and the high aspect ratio zinc oxide more remarkably than in the case of using only the low aspect ratio zinc oxide.

In the present invention, the average aspect ratio represents (average particle size)/(average thickness). The average particle size in the present invention means an average primary particle size, and can be determined, for example, as the arithmetic mean of the long diameter and the short diameter of a particle by image analysis with a transmission electron microscope. The average thickness can be determined, for example, as the arithmetic mean of the thicknesses of 20 particles by image analysis with a transmission electron microscope.

The aspect ratio of zinc oxide generally has a distribution. Therefore, when the distribution of the aspect ratio of zinc oxide included in the cosmetic, according to the present invention, including a combination of low aspect ratio zinc oxide and high aspect ratio zinc oxide is observed, for example, by image analysis, two maxima are generally present at a position where the aspect ratio is less than 4 and a position where the aspect ratio is 4 or more.

[Low Aspect Ratio Zinc Oxide]

The cosmetic according to the present invention includes low aspect ratio zinc oxide having an average aspect ratio of less than 4.

The particle shape of the low aspect ratio zinc oxide is not particularly limited, and examples of the particle shape include spherical, plate, and crushed shapes.

The low aspect ratio zinc oxide preferably has an average particle size of 20 to 60 nm, and more preferably 30 to 55 nm.

The low aspect ratio zinc oxide is not particularly limited as long as it is hydrophobic zinc oxide generally used as a cosmetic, and is preferably hydrophobic zinc oxide.

Hydrophobic zinc oxide is formed by hydrophobizing the surface. Hydrophobizing the surface improves the water resistance and the dispersibility in oil. As a method of hydrophobizing, a treatment with an alkylalkoxysilane is preferable for hydrophobizing the surface. Examples of the alkylalkoxysilane include octyltriethoxysilane and octyltrimethoxysilane, and a treatment with octyltriethoxysilane is preferable.

The amount of the low aspect ratio zinc oxide blended is preferably 1 to 50 mass % and more preferably 3 to 30 mass % with respect to the total amount of the cosmetic of the present invention.

[High Aspect Ratio Zinc Oxide]

The cosmetic according to the present invention includes high aspect ratio zinc oxide having an average aspect ratio of 4 or more.

The particle shape of the high aspect ratio zinc oxide is preferably a plate shape, and more preferably a hexagonal plate shape.

The high aspect ratio zinc oxide preferably has an average particle size of 30 to 5000 nm, and more preferably 50 to 4000 nm.

The high aspect ratio zinc oxide may be hydrophobic zinc oxide or non-hydrophobic zinc oxide. Hydrophobic zinc oxide is formed by hydrophobizing the surface in the same manner as described above. The term “non-hydrophobic zinc oxide” refers to zinc oxide whose surface is not hydrophobized, and non-hydrophobic zinc oxide is hydrophilic.

In a case where the high aspect ratio zinc oxide is hydrophobic zinc oxide, the surface-hydrophobizing treatment is not particularly limited, and examples of the method of hydrophobizing include a treatment with hydrogen dimethicone, a treatment with dimethicone, a treatment with octyltriethoxysilane, a treatment with fatty acid dextrin, and a treatment with perfluoroalkyl phosphoric acid.

In a case where the cosmetic according to the present invention is a water-in-oil or oil-in-water emulsion cosmetic, both the low aspect ratio zinc oxide and the high aspect ratio zinc oxide are preferably present in the oil phase.

[Dispersant]

The cosmetic according to the present invention preferably further includes a dispersant. The dispersant disperses zinc oxide in the cosmetic, and preferably disperses zinc oxide in the oil phase. Examples of the dispersant include isostearic acid, bis-butyldimethicone polyglyceryl-3, and sorbitan sesquiisostearate, and isostearic acid is preferable.

The amount of the dispersant blended is preferably 0.1 to 5 mass % and more preferably 0.3 to 3 mass % with respect to the total amount of the cosmetic.

[Ultraviolet Absorber]

The cosmetic according to the present invention can include an ultraviolet absorber. The ultraviolet absorber can be at least one selected from ultraviolet absorbers conventionally blended in sunscreen cosmetics commonly.

Specific examples of the UVA absorber include methylenebis-benzotriazolyl tetramethylbutylphenol, t-butyl nnethoxydibenzoylnnethane, diethylamino hydroxybenzoyl hexyl benzoate, 2-ethylhexyl dinnethoxybenzylidene dioxoinnidazolidine propionate, terephthalylidene dicamphor sulfonic acid, and bis-ethylhexyloxyphenol methoxyphenyl triazine.

Specific examples of the UVB absorber include ethylhexyl nnethoxycinnamate, ethylhexyl triazone, octocrylene, dinnethicodiethylbenzalnnalonate, polysilicone-15, oxybenzone-3, phenylbenzimidazole sulfonic acid, homosalate, drometrizole trisiloxane, and ethylhexyl salicylate.

[Surfactant]

The cosmetic according to the present invention can include a surfactant. Examples of the surfactant include anionic surfactants, cationic surfactants, amphoteric surfactants, and nonionic surfactants.

[Metal Salt]

The cosmetic according to the present invention can include a metal salt selected from the group consisting of water-soluble aluminium compounds, water-soluble zirconium compounds, and zinc sulfonate compounds. The metal salt is, for example, selected from the group consisting of aluminium chloride, aluminium sulfate, aluminium chlorohydrate, aluminium chlorohydroxy allantoinate, aluminium dihydroxy allantoinate, bromohydroxyaluminum, aluminium acetate, aluminium lactate, aluminium zirconium chlorohydrate, zirconium chlorohydrate, aluminum potassium sulfate, and zinc paraphenolsulfonate, more preferably selected from the group consisting of aluminium chlorohydrate and zinc paraphenolsulfonate, and is preferably aluminium chlorohydrate or zinc paraphenolsulfonate.

[Pigment]

The cosmetic according to the present invention can include a pigment. Examples of the pigment include inorganic white pigments, inorganic red-based pigments, inorganic brown-based pigments, inorganic yellow-based pigments, inorganic black-based pigments, inorganic violet-based pigments, inorganic green-based pigments, inorganic blue-based pigments, pearl pigments, metal powder pigments, organic pigments, and natural pigments. Specific examples thereof include talc, mica, and sericite.

[Fine Particulate Metal Oxide]

The cosmetic according to the present invention can include a fine particulate oxide other than the above. Examples of the fine particulate oxide include fine particulate iron oxide, fine particulate titanium oxide, and cerium oxide.

[Another Powder]

The cosmetic according to the present invention can include a powder other than the pigments and the particulate metal oxides. Examples of another powder include silica, a starch powder, and a cellulose powder.

The amount of another powder blended is preferably 10 mass % or less and more preferably 5 mass % or less with respect to the total amount of the cosmetic. The cosmetic according to the present invention that does not include another powder (0 mass %) is also one aspect of the present invention.

Examples of an optional component other than the above include components commonly used in cosmetics, such as moisturizers, antioxidants, preservatives, alcohols, oil thickeners, aqueous phase thickeners, oily components such as silicone oils and ester oils, whitening agents, and extracts.

The cosmetic according to the present invention can include water. The amount of water blended is preferably 20 to 80 mass % and more preferably 30 to 60 mass % with respect to the total amount of the cosmetic.

The cosmetic according to the present invention can be preferably used as, for example, a makeup cosmetic such as a makeup base or a foundation, a base cosmetic, or a sunscreen cosmetic.

The form of the cosmetic according to the present invention is not particularly limited, but a water-in-oil or oil-in-water emulsion cosmetic is preferable, and a water-in-oil emulsion cosmetic is more preferable.

The cosmetic of the present invention can be produced in accordance with a conventional method. In a case where the cosmetic according to the present invention is a water-in-oil or oil-in-water emulsion cosmetic, the method of emulsification is not particularly limited.

### EXAMPLES

The present invention will be described specifically with reference to the following Examples, but the present invention is not limited to these Examples. The content is shown by mass % with respect to the total amount unless otherwise specified.

### Examples 1 to 5 and Comparative Examples 1 and 2

Cosmetics of Examples 1 to 5 and Comparative Examples 1 and 2 were prepared at a blending proportion shown in Table 1, and their effects of solidifying sebum were evaluated.

TABLE 1

| | | | | | | Comparative | |
|---|---|---|---|---|---|---|---|
| | Example | | | | | Example | |
| | 1 | 2 | 3 | 4 | 5 | 1 | 2 |
| Low aspect ratio zinc oxide A | 6 | 6 | 4 | 6.5 | 5 | 6 | 6 |
| Low aspect ratio zinc oxide B | — | — | — | — | — | 1 | — |
| Low aspect ratio zinc oxide C | — | — | — | — | — | — | 1 |
| High aspect ratio zinc oxide A | 1 | — | 3 | 0.5 | 2 | — | — |
| High aspect ratio zinc oxide B | — | 1 | — | — | — | — | — |
| Isostearic acid | 0.5 | 0.5 | 0.5 | 0.5 | 0.5 | 0.5 | 0.5 |
| Ethylhexyl methoxycinnamate | 7 | 7 | 7 | 7 | 7 | 7 | 7 |
| Bis-ethylhexyloxyphenol | 0.5 | 0.5 | 0.5 | 0.5 | 0.5 | 0.5 | 0.5 |
| methoxyphenyl triazine | | | | | | | |
| Dimethicone | 27 | 27 | 27 | 27 | 27 | 27 | 27 |
| Caprylyl methicone | 2.5 | 2.5 | 2.5 | 2.5 | 2.5 | 2.5 | 2.5 |
| Diphenylsiloxy phenyl trimethicone | 1.5 | 1.5 | 1.5 | 1.5 | 1.5 | 1.5 | 1.5 |
| PEG-9 polydimethylsiloxyethyl | 0.9 | 0.9 | 0.9 | 0.9 | 0.9 | 0.9 | 0.9 |
| dimethicone | | | | | | | |
| Disteardimonium hectorite | 0.3 | 0.3 | 0.3 | 0.3 | 0.3 | 0.3 | 0.3 |
| Water | 45.8 | 45.8 | 45.8 | 45.8 | 45.8 | 45.8 | 45.8 |
| Ethanol | 6.5 | 6.5 | 6.5 | 6.5 | 6.5 | 6.5 | 6.5 |
| Phenoxyethanol | 0.5 | 0.5 | 0.5 | 0.5 | 0.5 | 0.5 | 0.5 |
| Total | 100 | 100 | 100 | 100 | 100 | 100 | 100 |
| Difference determined by gloss meter | 0.77 | 1.67 | 1.47 | 0.87 | 0.60 | 2.33 | 2.40 |

In the table:
Low aspect ratio zinc oxide A: “MZX-3040TS” (manufactured by TAYCA CORPORATION), treated with octyltriethoxysilane, amorphous, average particle size: 35 nm,
Low aspect ratio zinc oxide B: “XZ-300F-LP” (manufactured by SAKAI CHEMICAL INDUSTRY CO., LTD.), treated with hydrogen dimethicone, hexagonal plate shape, average particle size: 300 nm, average aspect ratio: 3,
Low aspect ratio zinc oxide C: “XZ-1000F-LP” (manufactured by SAKAI CHEMICAL INDUSTRY CO., LTD.), treated with hydrogen dimethicone, hexagonal plate shape, average particle size: 1000 nm, average aspect ratio: 3.4,
High aspect ratio zinc oxide A: “XZ 1000FST” (manufactured by SAKAI CHEMICAL INDUSTRY CO., LTD.), not hydrophobized, hexagonal plate shape, average particle size: 1000 nm, average aspect ratio: 10 to 12.5,
High aspect ratio zinc oxide C: “XZ-3000F-LP” (manufactured by SAKAI CHEMICAL INDUSTRY CO., LTD.), treated with hydrogen dimethicone, hexagonal plate shape, average particle size: 3000 nm, average aspect ratio: 4.

[Method for Evaluating Effect of Solidifying Sebum]

Each of the cosmetics of Examples and Comparative Examples was applied in an amount of 1 mg/cm 2 to an artificial leather (“SUPPLALE”, manufactured by IDEATEX JAPAN Co., Ltd.), and 15 minutes after the application, the gloss value at 60° was measured using a gloss meter “VG-2000” (manufactured by NIPPON DENSHOKU INDUSTRIES CO., LTD.). Oleic acid was further applied in an amount of 0.5 mg/cm², and 30 minutes after the application of oleic acid, the gloss value was measured in the same manner. The artificial leather was allowed to stand on a hot plate at 37° C. until the measurement 30 minutes after the application of oleic acid. In each of Examples and Comparative Examples, the above-described measurements were each performed three times, and the average values each obtained by the three measurements were used to calculate the difference determined by the gloss meter (gloss value after application of oleic acid—gloss value before application of oleic acid). Table 1 shows the obtained results.

In the case of using the same manner as described above except that no cosmetic was applied to the artificial leather, the difference determined by the gloss meter was 2.5.

From the results in Table 1, it has been found that a cosmetic including low aspect ratio zinc oxide and high aspect ratio zinc oxide has a high effect of solidifying sebum.

1. A cosmetic comprising:
low aspect ratio zinc oxide having an average aspect ratio of less than 4; and high aspect ratio zinc oxide having an average aspect ratio of 4 or more, wherein
an amount of the high aspect ratio zinc oxide blended is 1 to 80 mass % with respect to a total amount of the high aspect ratio zinc oxide and the low aspect ratio zinc oxide.

2. The cosmetic according to claim 1, wherein the low aspect ratio zinc oxide is zinc oxide surface-treated with an alkylalkoxysilane.

3. The cosmetic according to claim 1, wherein the low aspect ratio zinc oxide is zinc oxide surface-treated with octyltriethoxysilane.

4. The cosmetic according to claim 1, wherein the low aspect ratio zinc oxide has an average particle size of 20 to 60 nm.

5. The cosmetic according to claim 1, wherein the low aspect ratio zinc oxide has an average particle size of 30 to 55 nm.

6. The cosmetic according to claim 1, wherein the high aspect ratio zinc oxide is a plate-shaped crystal.

7. The cosmetic according to claim 1, further comprising a dispersant.

8. The cosmetic according to claim 1, being a water-in-oil or oil-in-water emulsion cosmetic.
